(19) United States
(12) Patent Application Publication
(10) Pub. No.: US 2010/0022613 A1
(43) Pub. Date: Jan. 28, 2010

(54) Compounds Having CRTH2 Antagonist Activity

(75) Inventors: Richard Edward Armer, Abingdon (GB); Graham Michael Wynne, Abingdon (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: Oxagen Limited, Oxon (GB)

(21) Appl. No.: 12/357,792

(22) Filed: Jan. 22, 2009

(30) Foreign Application Priority Data
Jan. 22, 2008 (GB) GB0801132.2
Jan. 30, 2008 (GB) GB0801671.9

(51) Int. Cl. A61K 31/405; C07D 209/04

(52) U.S. Cl. 514/415; 548/491

(57) ABSTRACT

Compounds of general formula (I)

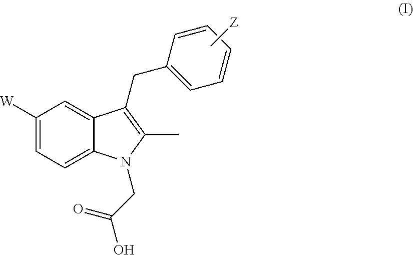

W is chloro or fluoro;
Z is a —SO₂YR¹ group wherein R¹ is C₃-C₈ heterocyclyl, aryl or heteroaryl any of which may optionally be substituted with one or more substituents selected from halo, —CN, —C₁-C₆ alkyl, —SOR³, —SO₂R³, —SO₂N(R²)₂, —N(R²)₂, —NR²C(O)R³, —CO₂R², —CONR²R³, —NO₂, —OR², —SR², —O(CH₂)ₚOR², and —O(CH₂)ₚO(CH₂)_{q}OR² wherein
   each R² is independently hydrogen, —C₁-C₆ alkyl, —C₃-C₈ cycloalkyl, aryl or heteroaryl;
   each R³ is independently, —C₁-C₆ alkyl, —C₃-C₈ cycloalkyl, aryl or heteroaryl;
   p and q are each independently an integer from 1 to 3;
Y is a straight or branched C₁-C₄ alkylene chain;
and their pharmaceutically acceptable salts, hydrates, solvates, complexes or prodrugs are useful in orally administrable compositions for the treatment of allergic diseases such as asthma, allergic rhinitis and atopic dermatitis.

The present invention relates to compounds which are useful as pharmaceuticals, to methods for preparing these compounds, compositions containing them and their use in the treatment and prevention of allergic diseases such as asthma, allergic rhinitis and atopic dermatitis and other inflammatory diseases mediated by prostaglandin D₂ (PGD₂) or other agonists acting at the CRTH2 receptor on cells including eosinophils, basophils and Th2 lymphocytes.

PGD₂ is an eicosanoid, a class of chemical mediator synthesised by cells in response to local tissue damage, normal stimuli or hormonal stimuli or via cellular activation pathways. Eicosanoids bind to specific cell surface receptors on a wide variety of tissues throughout the body and mediate various effects in these tissues. PGD₂ is known to be produced by mast cells, macrophages and Th2 lymphocytes and has been detected in high concentrations in the airways of asthmatic patients challenged with antigen (Murray et al., (1986), N. Engl. J. Med. 315: 800-804). Instillation of PGD₂ into airways can provoke many features of the asthmatic response including bronchoconstriction (Hardy et al., (1984) N. Engl. J. Med. 311: 209-213; Sampson et al., (1997) Thorax 52: 513-518) and eosinophil accumulation (Emery et al., (1989) J. Appl. Physiol. 67: 959-962).

The potential of exogenously applied PGD₂ to induce inflammatory responses has been confirmed by the use of transgenic mice overexpressing human PGD₂ synthase which exhibit exaggerated eosinophilic lung inflammation and Th2 cytokine production in response to antigen (Fujitani et al, (2002) J. Immunol. 168: 443-449).

The first receptor specific for PGD₂ to be discovered was the DP receptor which is linked to elevation of the intracellular levels of cAMP. However, PGD₂ is thought to mediate much of its proinflammatory activity through interaction with a G protein-coupled receptor termed CRTH2 (chemoattractant receptor-homologous molecule expressed on Th2 cells) which is expressed by Th2 lymphocytes, eosinophils and basophils (Hirai et al., (2001) J. Exp. Med. 193: 255-261, and EP0851030 and EP-A-1211513 and Bauer et al., EP-A-1170594). It seems clear that the effect of PGD₂ on the activation of Th2 lymphocytes and eosinophils is mediated through CRTH2 since the selective CRTH2 agonists 13,14 dihydro-15-keto-PGD₂ (DK-PGD₂) and 15R-methyl-PGD₂ can elicit this response and the effects of PGD₂ are blocked by an anti-CRTH2 antibody (Hirai et al., 2001; Monneret et al., (2003) J. Pharmacol. Exp. Ther. 304: 349-355). In contrast, the selective DP agonist BW245C does not promote migration of Th2 lymphocytes or eosinophils (Hirai et al., 2001; Gervais et al., (2001) J. Allergy Clin. Immunol. 108: 982-988). Based on this evidence, antagonising PGD₂ at the CRTH2 receptor is an attractive approach to treat the inflammatory component of Th2-dependent allergic diseases such as asthma, allergic rhinitis and atopic dermatitis.

EP-A-1170594 suggests that the method to which it relates can be used to identify compounds which are of use in the treatment of allergic asthma, atopic dermatitis, allergic rhinitis, autoimmune, reperfusion injury and a number of inflammatory conditions, all of which are mediated by the action of PGD₂ or other agonists at the CRTH2 receptor.

Compounds which bind to CRTH2 are taught in WO-A-03066046 and WO-A-03066047. These compounds are not new but were first disclosed, along with similar compounds, in GB 1356834, GB 1407658 and GB 1460348, where they were said to have anti-inflammatory, analgesic and antipyretic activity. WO-A-03066046 and WO-A-03066047 teach that the compounds to which they relate are modulators of CRTH2 receptor activity and are therefore of use in the treatment or prevention of obstructive airway diseases such as asthma, chronic obstructive pulmonary disease (COPD) and a number of other diseases including various conditions of bones and joints, skin and eyes, GI tract, central and peripheral nervous system and other tissues as well as allograft rejection. These compounds are all indole derivatives with an acetic acid substituent at the 3-position of the indole ring.

PL 65781 and JP 43-24418 also relate to indole-3 acetic acid derivatives which are similar in structure to indomethacin and, like indomethacin, are said to have anti-inflammatory and antipyretic activity. Thus, although this may not have been appreciated at the time when these documents were published, the compounds they describe are COX inhibitors, an activity which is quite different from that of the compounds of the present invention. Indeed, COX inhibitors are contraindicated in the treatment of many of the diseases and conditions, for example asthma and inflammatory bowel disease for which the compounds of the present invention are useful, although they may sometimes be used to treat arthritic conditions.

There is further prior art which relates to indole-1-acetic acid compounds, although these are not described as CRTH2 antagonists. For example WO-A-9950268, WO-A-0032180, WO-A-0151849 and WO-A-0164205 all relate to compounds which are indole-l-acetic acid derivatives but these compounds are said to be aldose reductase inhibitors useful in the treatment of diabetes mellitus (WO-A-9950268, WO-A-0032180 and WO-A-0164205) or hypouricemic agents (WO-A-0151849). There is no suggestion in any of these documents that the compounds would be useful for the treatment of diseases and conditions mediated by PGD₂ or other CRTH2 receptor agonists.

U.S. Pat. No. 4,363,912 relates to indole-1-alkyl carboxylic acid derivatives (including indole-1-acetic acid analogues) which are said to be inhibitors of thromboxane synthetase and to be useful in the treatment of conditions such as thrombosis, ischaemic heart disease and stroke. In contrast to the compounds of the present invention (which are all indole-1-acetic acid derivatives) the preferred compounds within U.S. Pat. No. 4,363,912 are 3-(indol-1-yl)-propionic acid derivatives.

WO-A-9603376 relates to compounds which are said to be sPLA₂ inhibitors which are useful in the treatment of bronchial asthma and allergic rhinitis. These compounds all have amide or hydrazide substituents in place of the carboxylic acid derivative of the compounds of the present invention.

JP 2001247570 relates to a method of producing a 3-benzothiazolyhnethyl indole acetic acid, which is said to be an aldose reductase inhibitor.

U.S. Pat. No. 4,859,692 relates to compounds which are said to be leukotriene antagonists useful in the treatment of conditions such as asthma, hay fever and allergic rhinitis as well as certain inflammatory conditions such as bronchitis, atopic and ectopic eczema. Some of the compounds of this document are indole-1-acetic acids but the same authors, in J. Med. Chem., 33, 1781-1790 (1990), teach that compounds with an acetic acid group on the indole nitrogen do not have significant peptidoleukotriene activity.

U.S. Pat. No. 4,273,782 is directed to indole-1-acetic acid derivatives which are said to be useful in the treatment of conditions such as thrombosis, ischaemic heart disease, stroke, transient ischaemic attack, migraine and the vascular complications of diabetes. There is no mention in the document of conditions mediated by the action of PGD₂ or other agonists at the CRTH2 receptor.

U.S. Pat. No. 3,557,142 relates to 3-substituted-1-indole carboxylic acids and esters which are said to be useful in the treatment of inflammatory conditions.

WO-A-03/097598 relates to compounds which are CRTH2 receptor antagonists. They do not have an aromatic substituent at the indole-3 position.

Cross et al., J. Med. Chem. 29, 342-346 (1986) relates to a process for preparing indole-1-acetic acid derivatives from the corresponding esters. The compounds to which it relates are said to be inhibitors of thromboxane synthetase.

EP-A-0539117 relates to indole-1-acetic acid derivatives which are leukotriene antagonists.

US 2003/0153751 relates to indole-1-acetic acid derivatives which are sPLA₂ inhibitors. However, all of the exemplified compounds have bulky substituents at the 2- and 5-positions of the indole system and are therefore very different from the compounds of the present invention.

US 2004/011648 discloses indole-1-acetic acid derivatives which are inhibitors of PAI-1. There is no suggestion that the compounds might have CRTH2 antagonist activity.

WO 2004/058164 relates to compounds which are said to be asthma and allergic inflammation modulators. The only compounds for which activity is demonstrated are entirely different in structure from the indole-1-acetic acid derivatives of the present invention.

Compounds which bind to the CRTH2 receptor are disclosed in WO-A-03/097042 and WO-A-03/097598. These compounds are indole acetic acids but in WO-A-03/097042 the indole system is fused at the 2-3 positions to a 5-7 membered carbocyclic ring. In WO-A-03/097598 there is a pyrrolidine group at the indole 3-position.

WO-A-03/101981, WO-A-03/101961 and WO-A-2004/007451 all relate to indole-1-acetic acid derivatives which are said to be CRTH2 antagonists but which differ in structure from the compounds of general formula (I) because there is no spacer or an —S— or —SO₂— group attached to the indole 3-position in place of the CH₂ group of the compounds of the present invention as described below.

WO-A-2005/019171 also describes indole-1-acetic acid derivatives which are said to be CRTH2 antagonists and which are said to be useful for the treatment of various respiratory diseases. These compounds all have a substituent which is linked to the indole-3 position by an oxygen spacer.

WO-A-2005/094816 again describes indole-1-acetic acid compounds, this time with an aliphatic substituent at the 3-position of the indole ring. The compounds are said to be CRTH2 antagonists.

WO-A-2006/034419 relates to CRTH2 antagonist indole compounds which have a heterocyclic or heteroaromatic substituent directly linked to the 3-position of the indole ring system.

In our earlier application, WO-A-2005/044260, we describe compounds which are antagonists of PGD₂ at the CRTH2 receptor. These compounds are indole-1-acetic acid derivatives substituted at the 3-position with a group CR⁸R⁹, wherein R⁹ is hydrogen or alkyl and R⁸ is an aryl moiety which may be substituted with one or more substituents. The compounds described in this document are potent antagonists in vitro of PGD₂ at the CRTH2 receptor. However, we have found that when tested in vivo, the pharmacokinetic profile of some compounds is not optimal and their potency in the whole blood eosinophil shape change test, which gives an indication of the likely in vivo activity of the compounds, is often somewhat less than might have been expected from the in vitro binding results.

In another of our earlier applications, WO2006/095183, the indole-1-acetic acid derivatives are substituted at the 3-position with a 1-benzenesulfonyl-1H-pyrrol-2-ylmethyl group, where the phenyl group of the benzenesulfonyl moiety may be substituted. These compounds are extremely active CRTH2 antagonists but are rapidly metabolised as determined by incubation with human microsome preparations.

Our application WO2008/012511 also relates to CRTH2 antagonist compounds, this time to indole-1-acetic acid derivatives substituted at the 3-position with a 2-phenylsulfonylbenzyl group. It was found that the position of the phenylsulfonyl substituent had a significant effect on both the activity of the compounds and their pharmacokinetic profile.

The present invention relates to analogues of the compounds of WO2008/012511 in which the 2-phenylsulfonylbenzyl group is replaced by an aralkylsulfonylbenzyl group, a heteroarylalkylsulfonylbenzyl or a heterocyclylalkylsulfonylbenzyl group.

In the present invention there is provided a compound of general formula (I)

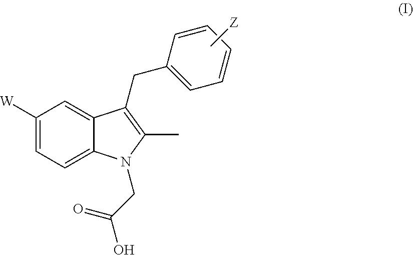

wherein

W is chloro or fluoro;

Z is a —SO₂YR¹ group wherein R¹ is C₃-C₈ heterocyclyl, aryl or heteroaryl any of which may optionally be substituted with one or more substituents selected from halo, —CN, —C₁-C₆ alkyl, —SOR³, —SO₂R³, —SO₂N(R²)₂, —N(R²)₂, —NR²C(O)R³, —CO₂R², —CONR²R³, —NO₂, —OR², —SR², —O(CH₂)ₚOR², and —O(CH₂)ₚO(CH₂)_{q}OR² wherein
each R² is independently hydrogen, —C₁-C₆ alkyl, —C₃-C₈ cycloalkyl, aryl or heteroaryl;
each R³ is independently, —C₁-C₆ alkyl, —C₃-C₈ cycloalkyl, aryl or heteroaryl;
p and q are each independently an integer from 1 to 3;

Y is a straight or branched C₁-C₄ alkylene chain;

or a pharmaceutically acceptable salt, hydrate, solvate, complex or prodrug thereof.

The compounds of the present invention have comparable in vitro CRTH2 binding to their 2-phenylsulfonylbenzyl analogues disclosed in WO2008/012511 but preferred compounds of the present invention have enhanced functional activity in inhibiting eosinophil activation. For example, Compound 1 (see below) has a Kᵢ in a CRTH2 ligand binding assay of 1 nM and also antagonises PGD₂-mediated activation of eosinophils with an IC₅₀ of 1 nM in whole blood.

The compounds of general formula (I) are antagonists at the CRTH2 receptor and are useful in the treatment of diseases and conditions which are mediated by PGD₂ or other agonists binding to the CRTH2 receptor. These include allergic diseases, asthmatic conditions and inflammatory diseases, examples of which are asthma, including allergic asthma, bronchial asthma, exacerbations of asthma and related allergic diseases caused by viral infection, particularly those exacerbations caused by rhinovirus and respiratory syncytial virus intrinsic, extrinsic, exercise-induced, drug-induced and dust-induced asthma, treatment of cough, including chronic cough associated with inflammatory and secretory conditions of the airways and iatrogenic cough, acute and chronic rhinitis, including rhinitis medicamentosa, vasomotor rhinitis, perennial allergic rhinitis, seasonal allergic rhinitis, nasal polyposis, acute viral infection including common cold, infection due to respiratory syncytial virus, influenza, coronavirus and adenovirus, atopic dermatitis, contact hypersensitivity (including contact dermatitis), eczematous dermatitis, phyto dermatitis, photo dermatitis, sebhorroeic dermatitis, dermatitis herpetiformis, lichen planus, lichen sclerosis et atrophica, pyoderma gangrenosum, skin sarcoid, discoid lupus erythematosus, pemphigus, pemphigoid, epidermolysis bullosa urticaria, angioedema, vasculitides, toxic erythemas, cutaneous eosinophilias, alopecia areata, male-pattern baldness, Sweet's syndrome, Weber-Christian syndrome, erythema multiforme, cellulitis, panniculitis, cutaneous lymphomas, non-melanoma skin cancer and other dysplastic lesions; blepharitis conjunctivitis, especially allergic conjunctivitis, anterior and posterior uveitis, choroiditis, autoimmune, degenerative or inflammatory disorders affecting the retina, ophthalmitis; bronchitis, including infectious and eosinophilic bronchitis, emphysema, bronchiectasis, farmer's lung, hypersensitivity pneumonitis, idiopathic interstitial pneumonias, complications of lung transplantation, vasculitic and thrombotic disorders of the lung vasculature, pulmonary hypertension, food allergies, gingivitis, glossitis, periodontitis, oesophagitis including reflux, eosinophilic gastroenteritis, proctitis, pruris ani, celiac disease, food-related allergies, inflammatory bowel disease, ulcerative colitis and Crohn's disease, mastocytosis and also other CRTH2-mediated diseases, for example autoimmune diseases such as hyper IgE syndrome, Hashimoto's thyroiditis, Graves' disease, Addison's disease, diabetes mellitus, idiopathic thrombocytopaenic purpura, eosinophilic paschiitis, antiphospholipid syndrome and systemic lupus erythematosus, AIDS, leprosy, Sezary syndrome, paraneoplastic syndrome, mixed and undifferentiated connective tissue diseases, inflammatory myopathies including dermatomyositis and polymyositis, polymalgia rheumatica, juvenile arthritis, rheumatic fever, vasculitides including giant cell arteritis, Takayasu's arteritis, Churg-Strauss syndrome, polyarteritis nodosa, microscopic polyarteritis, temporal arteritis, myasthenia gravis, acute and chronic pain, neuropathic pain syndromes, central and peripheral nervous system complications of malignant, infectious or autoimmune processes, low back pain, familial Mediterranean Fever, Muckle-Wells syndrome, Familial Hibernian fever, Kikuchi disease, psoriasis, acne, multiple sclerosis, allograft rejection, reperfusion injury, chronic obstructive pulmonary disease, as well as rheumatoid arthritis, Still's disease, ankylosing spondylitis, reactive arthritis, undifferentiated spondarthropathy, psoriatic arthritis, septic arthritis and other infection-related arthopathies and bone disorders and osteoarthritis; acute and chronic crystal-induced synovitis including urate gout, calcium pyrophosphate deposition disease, calcium paptite related tendon syndrome and synovial inflammation, Behcet's disease, primary and secondary Sjogren's syndrome systemic sclerosis and limited scleroderma; hepatitis, cirrhosis of the liver, cholecystitis, pancreatitis, nephritis, nephritic syndrome, cystitis and Hunner's ulcer, acute and chronic urethritis, prostatitis, epididymitis, oophoritis, salpingitis, vulvo-vaginitis, Peyronie's disease, erectile dysfunction, Alzheimer's disease and other dementing disorders; pericarditis, myocarditis, inflammatory and auto-immune cardiomyopathies including myocardial sarcoid, ischaemic reperfusion injuries, endocarditis, valvulitis, aortitis, phlebitis, thrombosis, treatment of common cancers and fibrotic conditions such as idiopathic pulmonary fibrosis including cryptogenic fibrosing alveolitis, keloids, excessive fibrotic scarring/adhesions post surgery, liver fibrosis including that associated with hepatitis B and C, uterine fibroids, sarcoidosis, including neurosarcoidosis, scleroderma, kidney fibrosis resulting from diabetes, fibrosis associated with RA, atherosclerosis, including cerebral atherosclerosis, vasculitis, myocardial fibrosis resulting from myocardial infarction, cystic fibrosis, restenosis, systemic sclerosis, Dupuytren's disease, fibrosis complicating anti-neoplastic therapy and chronic infection including tuberculosis and aspergillosis and other fungal infections, CNS fibrosis following stroke or the promotion of healing without fibrotic scarring.

The compounds are particularly effective when used for the treatment of allergic asthma, perennial allergic rhinitis, seasonal allergic rhinitis, atopic dermatitis, contact hypersensitivity (including contact dermatitis), conjunctivitis, especially allergic conjunctivitis, vernal keratoconjunctivitis and atopic keratoconjunctivitis, eosinophilic bronchitis, food allergies, eosinophilic gastroenteritis, inflammatory bowel disease, ulcerative colitis and Crohn's disease, mastocytosis and also other PGD2-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as rheumatoid arthritis, psoriatic arthritis, osteoarthritis and fibrotic diseases caused/exacerbated by Th2 immune responses, for example idiopathic pulmonary fibrosis and hypertrophic scars.

In the present specification “C₁-C₆ alkyl” refers to a straight or branched saturated hydrocarbon chain having one to six carbon atoms and optionally substituted with one or more halo substituents and/or with one or more C₃-C₇ cycloalkyl groups. Examples include methyl, ethyl, n-propyl, isopropyl, t-butyl, n-hexyl, trifluoromethyl, 2-chloroethyl, methylenecyclopropyl, methylenecyclobutyl, methylenecyclobutyl and methylenecyclopentyl.

The term “C₁-C₁₈ alkyl” has a similar meaning to the above except that it refers to a straight or branched saturated hydrocarbon chain having one to eighteen carbon atoms.

In the present specification “C₃-C₈ cycloalkyl” refers to a saturated carbocyclic group having three to eight ring atoms and optionally substituted with one or more halo substituents. Examples include cyclopropyl, cyclopentyl, cyclohexyl and fluorocyclohexyl.

The term “heterocyclyl” in the context of the specification refers to a saturated ring system having from 4 to 8 ring atoms, at least one of which is a heteroatom selected from N, O and S, and which is optionally substituted by one or more substituents chosen from halo and oxo. Examples of heterocyclyl groups include azetidinyl, piperidinyl; tetrahydrofuranyl, tetrahydropyranyl, dioxanyl, thiomorpholinyl, 1,1-dioxo-thiomorpholinyl, morpholinyl, pyrrolidinyl, 4,4-difluoropiperidinyl, piperizinyl, azepanyl, 1,4-diazepanyl, 1,4-oxazepanyl and azocanyl

In the present specification a “C₁-C₄ alkylene chain” refers to a saturated straight or branched carbon chain. Examples include —(CH₂)_{z}—, where z is an integer of 1 to 4, —CH(CH₃)—, —C(CH₃)₂—, —CH(CH₃)—CH₂—, —C(CH₃)₂—CH₂—, —CH₂—CH(CH₃)—CH₂—, and CH₂—C(CH₃)₂—CH₂—.

In the present specification, “halo” refers to fluoro, chloro, bromo or iodo.

The term “aryl” in the context of the present specification refer to a ring system with aromatic character having from 5 to 14 ring carbon atoms and containing up to three rings. Where an aryl group contains more than one ring, not all rings must be fully aromatic in character. Examples of aryl moieties are benzene, naphthalene, indane and indene.

The term “heteroaryl” in the context of the specification refer to a ring system with aromatic character having from 5 to 14 ring atoms, at least one of which is a heteroatom selected from N, O and S, and containing up to three rings. Where a heteroaryl group contains more than one ring, not all rings must be fully aromatic in character. Examples of heteroaryl groups include pyridine, pyrimidine, indole, benzofuran, benzimidazole and indolene.

General formula (I) as shown above is intended to include all isotopic variants, for example the hydrogen atoms of the molecule can be ¹H, ²H or ³H and the carbon atoms can be ¹²C or ¹⁴C.

Appropriate pharmaceutically and veterinarily acceptable salts of the compounds of general formulae (I) include basic addition salts such as sodium, potassium, calcium, aluminium, zinc, magnesium and other metal salts as well as choline, diethanolamine, ethanolamine, ethyl diamine, megulmine and other well known basic addition salts as summarised in Paulekuhn et al., (2007) J. Med. Chem. 50: 6665-6672 and/or known to those skilled in the art.

Salts which are not pharmaceutically or veterinarily acceptable may still be valuable as intermediates.

Prodrugs are any covalently bonded compounds which release the active parent drug according to general formula (I) in vivo. Examples of prodrugs include alkyl esters of the compounds of general formula (I), for example the esters of general formula (II) below.

In particularly suitable compounds of general formula (I), W is a fluoro substituent. In other suitable compounds of general formula (I) R¹ is a phenyl group which may be either unsubstituted or substituted as defined above. It is particularly suitable that R¹ is phenyl which is either unsubstituted or substituted with a single halo substituent, usually fluoro or chloro, which is generally at the 4-position of the phenyl group R¹.

Suitably, Y is methylene.

Among the most suitable compounds of the present invention are those in which the group Z is at the 4-position of the benzyl group that links to the indole. These compounds appear to be particularly effective in inhibiting eosinophil activation and are significantly more active than the analogues in which the group Z is at the 2- or 3-position.

This finding is particularly surprising in view of the teaching of WO2008/012511, which relates to compounds in which there is a 2-phenylsulfonylbenzyl substituent at the 3-position of an indole-1-acetic acid. This document demonstrates that these 2-phenylsulfonylbenzyl compounds have significantly greater CRTH2 antagonist activity in whole blood than their 3-phenylsulfonylbenzyl and 4-phenylsulfonylbenzyl analogues. In view of this teaching, it is particularly surprising that the most active compounds of the present invention are those in which the aralkylsulfonyl, heteroarylalkylsulfonyl or heterocyclylalkylsulfonyl substituent, Z, is at the 4-position of the phenyl group to which it is attached.

Example compounds of the present invention include:
2-(3-(4-(Benzylsulfonyl)benzyl)-5-fluoro-2-methyl-1H-indol-1-yl)acetic acid (Compound 1);
2-(3-(4-(4-Chlorobenzylsulfonyl)benzyl)-5-fluoro-2-methyl-1H-indol-1-yl)acetic acid (Compound 2);
2-(3-(3-(Benzylsulfonyl)benzyl)-5-fluoro-2-methyl-1H-indol-1-yl)acetic acid (Compound 3);
2-(5-Fluoro-3-(3-(4-fluorobenzylsulfonyl)benzyl)-2-methyl-1H-indol-1-yl)acetic acid (Compound 4);
2-(3-(2-(Benzylsulfonyl)benzyl)-5-fluoro-2-methyl-1H-indol-1-yl)acetic acid (Compound 5);
2-(3-(4-(4-Fluorobenzylsulfonyl)benzyl)-5-fluoro-2-methyl-1H-indol-1-yl)acetic acid (Compound 6);
or the C₁-C₆ alkyl, aryl, (CH₂)ₘOC(═O)C₁-C₆alkyl, ((CH₂)ₘO)ₙCH₂CH₂X, (CH₂)ₘN(R⁵)₂ or CH((CH₂)ₘO(C═O)R⁶)₂ ester thereof;
   m is 1 or 2;
   n is 1-4;
   X is OR⁵ or N(R⁵)₂;
   R⁵ is hydrogen or methyl;
   R⁶ is C₁-C₁₈ alkyl.

Compounds 1, 2 and 6 are particularly active example compounds of the present invention and all of these compounds have the Z substituent at the 4-position of the phenyl group to which it is attached. Compounds 2 and 6 also have a 4-halo substituent at the 4-position of the phenyl group R¹, while in Compound 1, the phenyl group R¹ is unsubstituted.

In a further aspect of the present invention, there is provided a compound of general formula (II):

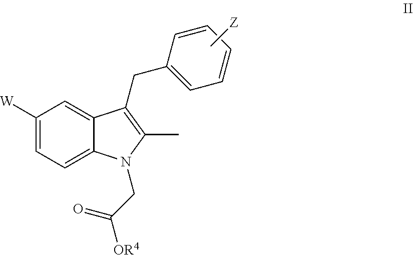

wherein W and Z are as defined for general formula (I); and

R⁴ is C₁-C₆ alkyl, C₁-C₆ alkyl substituted with aryl, aryl, (CH₂)ₘOC(═O)C₁-C₆alkyl, ((CH₂)ₘO)ₙCH₂CH₂X, (CH₂)ₘN(R⁵)₂ or CH((CH₂)ₘO(C═O)R⁶)₂;
m is 1 or 2;
n is 1-4;
X is OR⁵ or N(R⁵)₂;
R⁵ is hydrogen or methyl;
R⁶ is C₁-C₁₈ alkyl;

or a pharmaceutically acceptable salt, hydrate, solvate, complex or prodrug thereof.

Compounds of general formula (II) are novel and may be used as prodrugs for compounds of general formula (I). When the compound of general formula (II) acts as a prodrug, it is later transformed to the drug by the action of an esterase in the blood or in a tissue of the patient.

Examples of particularly suitable R⁴ groups when the compound of general formula (II) is used as a prodrug include:

methyl, ethyl, propyl, phenyl, —O(CH₂)₂O(CH₂)₂OR⁵, —O(CH₂)₂O(CH₂)₂O(CH₂)₂OR⁵, —O(CH₂)₂O(CH₂)₂NR⁵₂, —O(CH₂)₂O(CH₂)₂O(CH₂)₂NR⁵₂, —CH₂OC(═O)tBu, —CH₂CH₂N(Me)₂, —CH₂CH₂NH₂ or —CH(CH₂O(C═O)R⁶)₂ wherein R⁵ and R⁶ are as defined above.

In addition to their use as prodrugs, compounds of formula (II) wherein R⁴ is C₁-C₆ alkyl or benzyl may be used in a process for the preparation of a compound of general formula (I), the process comprising reacting the compound of general formula (II) with a base such as sodium hydroxide or lithium hydroxide. The reaction may take place in an aqueous solvent or an organic solvent or a mixture of the two. A typical solvent used for the reaction is a mixture of tetrahydrofuran and water. This reaction is described in detail for example compounds in Procedure F of the examples below.

Compounds of general formula (II) may be prepared from compounds of general formula (III):

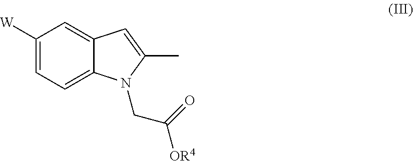

wherein W and R⁴ are as defined in general formula (II); by reaction with an aldehyde of general formula (IV):

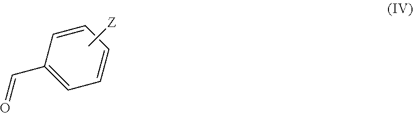

wherein Z is SO₂YR¹ as defined for general formula (I). The reaction may be carried out in the presence of trifluoroacetic acid and triethylsilane in a polar organic solvent such as dichloromethane and typically at room temperature (15 to 25° C.). An example of this reaction is described in Procedure E of the examples below.

Procedures for the preparation of compounds of general formula (III) are known to those skilled in the art and in general involve alkylation of a 5-halo-2-methylindole derivative at the 1-position with an alpha-bromoacetate derivative or related alkylating agent.

Compounds of general formula (IV) may be prepared by the oxidation of a compound of general formula (V)

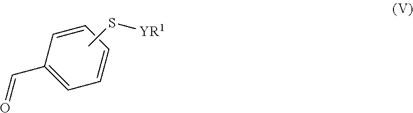

where Y and R¹ are as defined in general formula (I).

In some cases, the conversion may be achieved by protecting the compound of general formula (V) as an acetal of general formula (VI):

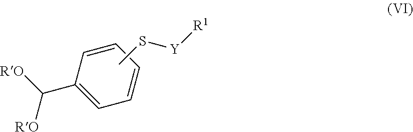

wherein Y and R¹ are as defined for general formula (I) and R′ represents C₁-C₆ alkyl, phenyl, benzyl or the two R′ groups may be linked together to form a cyclic acetal group, e.g. by condensation of a compound of general formula (V) with a C₂-C₆ diol such as propylene glycol or ethylene glycol.

Methods for forming acetals and their use as protecting groups are well known in the art and are described, for example in “Protecting Groups in Organic Synthesis”, Theodora W. Greene and Peter G. M. Wuts, published by John Wiley & Sons Inc. Typically, however, the acetal of general formula (VI) may be formed by reaction with trimethylorthoformate and p-toluene sulfonic acid followed by NaOR′, where R′ is as defined above, in the appropriate alcoholic solvent. For example when R′ is methyl, the reaction may be carried out using sodium methoxide in methanol. The reaction may initially be conducted in an anhydrous organic solvent such as methanol and under an inert atmosphere, typically nitrogen. The reaction is described in detail in Procedure B set out below.

The compound of general formula (VI) may be oxidised to form a compound of general formula (VII):

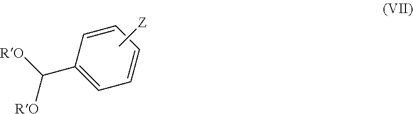

where Z is as defined in general formula (I) ) and R′ groups are as defined for general formula (VI);

by reaction with an oxidising agent such as 3-chloroperoxybenzoic acid (mCPBA) in a polar solvent such as dichloromethane. The reagents may be mixed at reduced temperature, for example −10 to 10° C. and the reaction mixture subsequently allowed to warm, for example to room temperature of about 15 to 25° C. This is described in detail for specific examples in Procedure C of the examples below.

The oxidation step is usually followed by the deprotection of the compound of general formula (VII) to form a compound of general formula (IV). The deprotection may be carried out by a standard method, for example by treating with aqueous acid, particularly sulfuric acid, followed by neutralisation with a base such as potassium carbonate as described in detail in Procedure D of the examples.

This method of forming the compound of general formula (IV) is particularly useful when the Z substituent is at the 2- or 4-position of the phenyl ring.

In some cases, particularly when Z is at the 3-position of the phenyl ring, protection is not necessary and the compound of general formula (IV) can be prepared directly from the compound of general formula (V) by direct oxidation, for example using mCPBA in dichloromethane as described above. This reaction is illustrated in Procedure J of the examples.

A compound of general formula (V) may be prepared by reacting a compound of general formula (VIII):

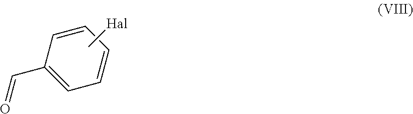

where Hal is fluoro, chloro or bromo;

with a compound of general formula (IX):

HSYR¹ (IX)

where Y and R¹ are as defined for general formula (I).

This substitution reaction may be carried out in the presence of a weak base such as potassium carbonate, in an organic solvent such as DMSO and under an inert atmosphere such as nitrogen. The reaction mixture may also be heated, for example at about 80 to 120° C., typically 100° C. The reaction is described in detail in Procedure A of the examples.

Compounds of general formulae (VIII) and (IX) are readily available or can be prepared by methods well known to those of skill in the art.

Alternatively, a compound of general formula (V) may be prepared from a compound of general formula (X):

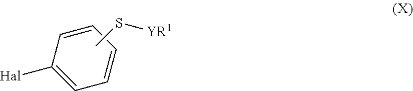

where Hal is F, Cl or Br and Y and R¹ are as defined in general formula (I);

by treatment with n-butyl lithium in THF followed by addition of DMF as described in Procedure H of the examples.

A compound of general formula (X) may be prepared from a compound of general formula (XI):

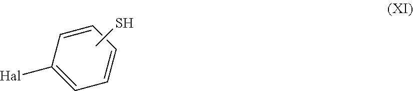

where Hal is as defined above;

by reaction with a compound of general formula (XII):

Hal-Y—R¹ (XII)

where Hal is F, Cl or Br and Y and R¹ are as defined in general formula (I).

The reaction may be carried out in a polar organic solvent such as acetonitrile and in the presence of a weak base such as cesium carbonate and is fully described in Procedure G of the examples.

Compounds of general formulae (XI) and (XII) are well known and are readily available or can be prepared by methods well known to those of skill in the art.

Compounds of general formula (I) are CRTH2 receptor antagonists and compounds of general formula (II) are prodrugs for compounds of general formula (I). Compounds of general formulae (I) and (II) are therefore useful in a method for the treatment of diseases and conditions mediated by PGD₂ or other agonists at the CRTH2 receptor, the method comprising administering to a patient in need of such treatment a suitable amount of a compound of general formula (I) or (II).

In a third aspect of the invention, there is provided a compound of general formula (I) or (II) for use in medicine, particularly for use in the treatment or prevention of diseases and conditions mediated by PGD₂ or other CRTH2 receptor agonists.

Furthermore, there is also provided the use of a compound of general formula (I) or (II) in the preparation of an agent for the treatment or prevention of diseases and conditions mediated by CRTH2 receptor agonists, particularly PGD₂.

As mentioned above, such diseases and conditions include allergic diseases, asthmatic conditions and inflammatory diseases, examples of which are asthma, including allergic asthma, bronchial asthma, intrinsic, extrinsic, exercise-induced, drug-induced and dust-induced asthma, treatment of cough, including chronic cough associated with inflammatory and secretory conditions of the airways and iatrogenic cough, acute and chronic rhinitis, including rhinitis medicamentosa, vasomotor rhinitis, perennial allergic rhinitis, seasonal allergic rhinitis, nasal polyposis, acute viral infection including common cold, infection due to respiratory syncytial virus, influenza, coronavirus and adenovirus, atopic dermatitis, contact hypersensitivity (including contact dermatitis), eczematous dermatitis, phyto dermatitis, photo dermatitis, sebhorroeic dermatitis, dermatitis herpetiformis, lichen planus, lichen sclerosis et atrophica, pyoderma gangrenosum, skin sarcoid, discoid lupus erythematosus, pemphigus, pemphigoid, epidermolysis bullosa urticaria, angioedema, vasculitides, toxic erythemas, cutaneous eosinophilias, alopecia areata, male-pattern baldness, Sweet's syndrome, Weber-Christian syndrome, erythema multiforme, cellulitis, panniculitis, cutaneous lymphomas, non-melanoma skin cancer and other dysplastic lesions; blepharitis conjunctivitis, especially allergic conjunctivitis, anterior and posterior uveitis, choroiditis, autoimmune, degenerative or inflammatory disorders affecting the retina, ophthalmitis; bronchitis, including infectious and eosinophilic bronchitis, emphysema, bronchiectasis, farmer's lung, hypersensitivity pneumonitis, idiopathic interstitial pneumonias, complications of lung transplantation, vasculitic and thrombotic disorders of the lung vasculature, pulmonary hypertension, food allergies, gingivitis, glossitis, periodontitis, oesophagitis including reflux, eosinophilic gastroenteritis, proctitis, pruris ani, celiac disease, food-related allergies, inflammatory bowel disease, ulcerative colitis and Crohn's disease, mastocytosis and also other CRTH2-mediated diseases, for example autoimmune diseases such as hyper IgE syndrome, Hashimoto's thyroiditis, Graves' disease, Addison's disease, diabetes mellitus, idiopathic thrombocytopaenic purpura, eosinophilic paschiitis, antiphospholipid syndrome and systemic lupus erythematosus, AIDS, leprosy, Sezary syndrome, paraneoplastic syndrome, mixed and undifferentiated connective tissue diseases, inflammatory myopathies including dermatomyositis and polymyositis, polymalgia rheumatica, juvenile arthritis, rheumatic fever, vasculitides including giant cell arteritis, Takayasu's arteritis, Churg-Strauss syndrome, polyarteritis nodosa, microscopic polyarteritis, temporal arteritis, myasthenia gravis, acute and chronic pain, neuropathic pain syndromes, central and peripheral nervous system complications of malignant, infectious or autoimmune processes, low back pain, familial Mediterranean Fever, Muckle-Wells syndrome, Familial Hibernian fever, Kikuchi disease, psoriasis, acne, multiple sclerosis, allograft rejection, reperfusion injury, chronic obstructive pulmonary disease, as well as rheumatoid arthritis, Still's disease, ankylosing spondylitis, reactive arthritis, undifferentiated spondarthropathy, psoriatic arthritis, septic arthritis and other infection-related arthopathies and bone disorders and osteoarthritis; acute and chronic crystal-induced synovitis including urate gout, calcium pyrophosphate deposition disease, calcium paptite related tendon syndrome and synovial inflammation, Behcet's disease, primary and secondary Sjogren's syndrome systemic sclerosis and limited scleroderma; hepatitis, cirrhosis of the liver, cholecystitis, pancreatitis, nephritis, nephritic syndrome, cystitis and Hunner's ulcer, acute and chronic urethritis, prostatitis, epididymitis, oophoritis, salpingitis, vulvo-vaginitis, Peyronie's disease, erectile dysfunction, Alzheimer's disease and other dementing disorders; pericarditis, myocarditis, inflammatory and auto-immune cardiomyopathies including myocardial sarcoid, ischaemic reperfusion injuries, endocarditis, valvulitis, aortitis, phlebitis, thrombosis, treatment of common cancers and fibrotic conditions such as idiopathic pulmonary fibrosis including cryptogenic fibrosing alveolitis, keloids, excessive fibrotic scarring/adhesions post surgery, liver fibrosis including that associated with hepatitis B and C, uterine fibroids, sarcoidosis, including neurosarcoidosis, scleroderma, kidney fibrosis resulting from diabetes, fibrosis associated with RA, atherosclerosis, including cerebral atherosclerosis, vasculitis, myocardial fibrosis resulting from myocardial infarction, cystic fibrosis, restenosis, systemic sclerosis, Dupuytren's disease, fibrosis complicating anti-neoplastic therapy and chronic infection including tuberculosis and aspergillosis and other fungal infections, and CNS fibrosis following stroke. The compounds are also of use in the promotion of healing without fibrotic scarring.

The compounds are particularly effective when used for the treatment or prevention of allergic asthma, perennial allergic rhinitis, seasonal allergic rhinitis, atopic dermatitis, contact hypersensitivity (including contact dermatitis), conjunctivitis, especially allergic conjunctivitis, vernal keratoconjunctivitis and atopic keratoconjunctivitis, eosinophilic bronchitis, food allergies, eosinophilic gastroenteritis, inflammatory bowel disease, ulcerative colitis and Crohn's disease, mastocytosis and also other PGD₂-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as rheumatoid arthritis, psoriatic arthritis, osteoarthritis and fibrotic diseases caused/exacerbated by Th2 immune responses, for example idiopathic pulmonary fibrosis and hypertrophic scars.

The compounds of general formula (I) or (II) must be formulated in an appropriate manner depending upon the diseases or conditions they are required to treat.

Therefore, in a further aspect of the invention there is provided a pharmaceutical composition comprising a compound of general formula (I) or (II) together with a pharmaceutical excipient or carrier. Other active materials may also be present, as may be considered appropriate or advisable for the disease or condition being treated or prevented.

The carrier, or, if more than one be present, each of the carriers, must be acceptable in the sense of being compatible with the other ingredients of the formulation and not deleterious to the recipient.

The formulations include those suitable for oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The route of administration will depend upon the condition to be treated but preferred compositions are formulated for oral, nasal, bronchial or topical administration.

The composition may be prepared by bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) or (II) in conjunction or association with a pharmaceutically or veterinarily acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, compounds of general formula (I) or (II) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Compounds of general formula (I) or (II) may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

Typically, the dose of the compound will be about 0.01 to 100 mg/kg; so as to maintain the concentration of drug in the plasma at a concentration effective to inhibit PGD₂ at the CRTH2 receptor. The precise amount of a compound of general formula (I) or (II) which is therapeutically effective, and the route by which such compound is best administered, is readily determined by one of ordinary skill in the art by comparing the blood level of the agent to the concentration required to have a therapeutic effect.

Compounds of general formula (I) or (II) may be used in combination with one or more active agents which are useful in the treatment of the diseases and conditions listed above, although these active agents are not necessarily inhibitors of PGD₂ at the CRTH2 receptor.

Therefore, the pharmaceutical composition described above may additionally contain one or more of these active agents.

There is also provided the use of a compound of general formula (I) or (II) in the preparation of an agent for the treatment of diseases and conditions mediated by CRTH2 receptor agonists, especially PGD₂, wherein the agent also comprises an additional active agent useful for the treatment of the same diseases and conditions.

These additional active agents may be other CRTH2 receptor antagonists or may have a completely different mode of action. They include existing therapies for allergic and other inflammatory diseases including:

Suplatast tosylate and similar compounds;

β2 adrenoreceptor agonists such as metaproterenol, isoproterenol, isoprenaline, albuterol, salbutamol, formoterol, salmeterol, indacaterol, terbutaline, orciprenaline, bitolterol mesylate and pirbuterol or methylxanthines such as theophylline and aminophylline, mast cell stabilisers such as sodium cromoglycate or muscarinic receptor antagonists such as tiotropium;

antihistamines, for example histamine H₁ receptor antagonists such as loratadine, cetirizine, desloratadine, levocetirizine, fexofenadine, astemizole, azelastine and chlorpheniramine or H₄ receptor antagonists;

α₁ and α₂ adrenoreceptor agonists such as propylhexedrine phenylephrine, phenylpropanolamine, pseudoephedrine, naphazoline hydrochloride, oxymetazoline hydrochloride, tetrahydrozoline hydrochloride, xylometazoline hydrochloride and ethylnorepinephrine hydrochloride;

modulators of chemokine receptor function, for example CCR1, CCR2, CCR2A, CCR2B, CCR3, CCR4, CCR5, CCR6, CCR7, CCR8, CCR9, CCR10 and CCR11 (for the C—C family) or CXCR1, CXCR2, CXCR3, CXCR4 and CXCR5 (for the C—X—C family) and CX₃CR1 for the C—X₃—C family;

Leukotriene antagonists such as montelukast and zafirlukast;

leukotriene biosynthesis inhibitors such as 5-lipoxygenase inhibitors or 5-lipoxygenase activating protein (FLAP) inhibitors such as zileuton, ABT-761, fenleuton, tepoxalin, Abbott-79175, N-(5-substituted)-thiophene-2-alkylsolfonamides, 2,6-di-tert-butylphenol hydrazones, methoxytetrahydropyrans such as ZD2138, SB-210661, pyridinyl-substituted-2-cyanonaphthalene compounds such as L-739010, 2-cyanoquinoline compounds such as L-746,530, indole and quinoline compounds such as MK-591, MK-886 and BAY x 1005;

Phosphdiesterase inhibitors, including PDE4 inhibitors such as roflumilast;

anti-IgE antibody therapies such as omalizumab;

anti-infectives such as fusidic acid (particularly for the treatment of atopic dermatitis);

anti-fungals such as clotrimazole (particularly for the treatment of atopic dermatitis);

immunosuppressants such as tacrolimus and particularly pimecrolimus in the case of inflammatory skin disease or alternatively FK-506, rapamycin, cyclosporine, azathioprine or methotrexate;

Immunotherapy agents including allergen immunotherapy such as Grazax;

corticosteroids such as prednisone, prednisolone, flunisolide, triamcinolone acetonide, beclomethasone dipropionate, budesonide, fluticasone propionate mometasone furoate and fluticasone furoate drugs which promote Th1 cytokine response such as interferons, TNF or GM-CSF.

CRTH2 antagonists may also be combined with therapies that are in development for inflammatory indications including:

other antagonists of PGD₂ acting at other receptors such as DP antagonists;

drugs that modulate cytokine production such as inhibitors of TNFα converting enzyme (TACE) anti-TNF monoclonal antibodies, TNF receptor immunoglobulin molecules, inhibitors of other TNF isoforms, non-selective COX-1/COX-2 inhibitors such as piroxicam, diclofenac, propionic acids such as naproxen, flubiprofen, fenoprofen, ketoprofen and ibuprofen, fenamates such as mefanamic acid, indomethacin, sulindac and apazone, pyrazolones such as phenylbutazone, salicilates such as aspirin; COX-2 inhibitors such as meloxicam, celecoxib, fofecoxib, valdecoxib and etoricoxib, low dose methotrexate, lefunomide, ciclesonide, hydroxychloroquine, d-penicillamine, auranofin or parenteral or oral gold;

drugs that modulate the activity of Th2 cytokines IL-4 and IL-5 such as blocking monoclonal antibodies and soluble receptors;

PPAR-γ agonists such as rosiglitazone; or with

anti-RSV antibodies such as Synagis (palivizumab) and agents that may be used to treat rhinovirus infection in the future e.g. intereferon-alpha, interferon-beta or other interferons.

In yet a further aspect of the invention, there is provided a product comprising a compound of general formula (I) or (II) and one or more of the agents listed above as a combined preparation for simultaneous, separate or sequential use in the treatment of a disease or condition mediated by the action of PGD₂ at the CRTH2 receptor.

In yet another aspect of the invention, there is provided a kit for the treatment of a disease or condition mediated by the action of PGD₂ at the CRTH2 receptor comprising a first container comprising a compound of general formula (I) or (II) and a second container comprising one or more of the active agents listed above.

The invention will now be described in greater detail with reference to the following non limiting examples.

In the Examples, the NMR spectra were obtained using a Bruker Advance II spectrometer operating at 300 MHz. All signals were referenced relative to residual protic solvent. HPLC-CAD-MS was performed on a Gilson 321 HPLC with detection performed by a ESA Corona CAD and a Finnigan AQA mass spectrometer operating in positive or negative ion electrospray ionisation mode. The HPLC column was a Phenomenex Gemini 15 C18 50×4.6 mm 3μ, with a mobile phase gradient between 100% 0.1% formic acid in water and 100% 0.1% formic acid in acetonitrile; with a total run time of either 6.5 or 12.5 minutes (the run time is stated in parenthesis).

### EXAMPLE 1

### Synthesis of Compounds of General Formula (I)

### Synthesis of 2-(3-(4-(benzylsulfonyl)benzyl)-5-fluoro-2-methyl-1H-indol-1-yl)-acetic acid (Compound 1) and Analogues

The synthesis was carried out according to Scheme 1

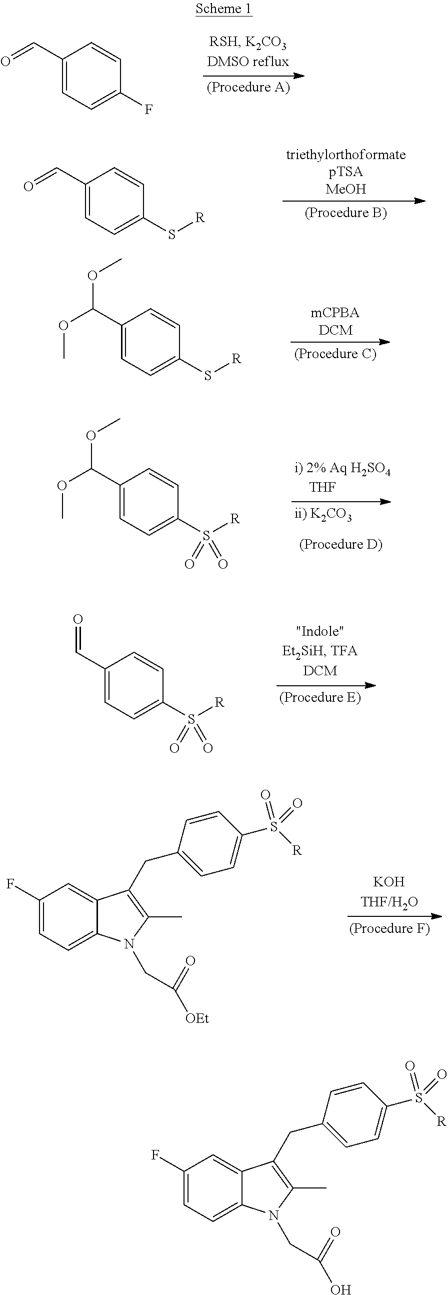

When the appropriate starting thiol is not commercially available, it may be prepared using Scheme 2 below.

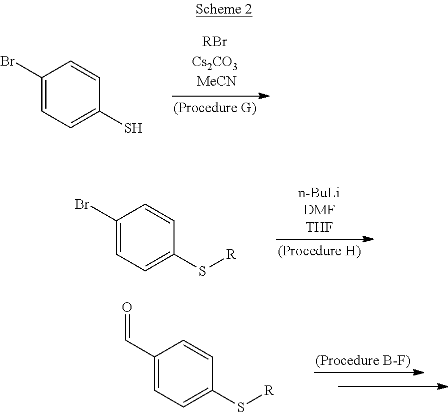

### Compound 1: 2-(3-(4-(Benzylsulfonyl)benzyl)-5-fluoro-2-methyl-1H-indol-1-yl)acetic acid

### Procedure A (S_{N}Ar): 4-(Benzylthio)benzaldehyde

To a solution of phenylmethanethiol (5.5 g, 44 mmol) in DMSO (20 ml) was added potassium carbonate (12 g, 87 mmol) and 4-fluorobenzaldehyde (5 g, 40 mmol). This was heated to 100° C. for about 4 hours. The solution was then cooled to room temperature, poured onto water, and extracted with ethyl acetate. The combined organic extracts were washed with brine, then dried over anhydrous magnesium sulfate, filtered and evaporated in vacuo. The crude 4-(benzylthio)benzaldehyde (9.07 g of a yellow solid, 40 mmol, 90%) was used directly without further purification.

¹H NMR (CDCl₃) 9.96 (1H, s, CHO), 7.83-7.77 (2H, m, Ar), 7.47-7.32 (7H, m, Ar) and 4.30 (2H, s, SCH₂).

### Procedure B. (Aldehyde Protection): Benzyl(4-(dimethoxymethyl)phenyl)sulfane

To a solution of the crude 4-(benzylthio)benzaldehyde (9.07 g, 40 mmol) in methanol (100 ml) was added trimethylorthoformate (4.85 ml, 44 mmol) and pTSA (900 mg, 5 mmol), and the resulting solution stirred at room temperature for about 18 hours. A solution of sodium methoxide in methanol (25% w/w, 1 ml) was then added, and the volatiles removed in vacuo. NMR analysis indicated complete conversion, so the resulting crude benzyl(4-(dimethoxymethyl)phenyl)sulfane was carried directly forward to Procedure C without further purification.

¹H NMR (CDCl₃) 7.48-7.24 (9H, m, Ar), 5.41 (1H, s, CH(OMe)₂), 4.18 (2H, s, SCH₂) and 3.36 (6H, s, CH(OMe)₂).

### Procedure C. (Oxidation): 1-(Benzylsulfonyl)-4-(dimethoxymethyl)benzene

Solid mCPBA (˜75%, 34 g, ˜150 mmol) was added slowly to the crude benzyl(4-(dimethoxymethyl)phenyl)sulfane from Procedure B (˜40 mmol) in DCM (100 ml) at 0° C., taking care to ensure that the internal temperature did not exceed 10° C. Once addition was complete, the ice bath was removed, and the solution allowed to warm to room temperature and stirred for approximately 60 hours. A solution of aqueous sodium metabisulfite (200 ml of the solution made from 90 g dissolved in 500 ml water) was then added, and the biphasic mixture was stirred vigorously for 2 hours. The solution was then neutralised with 1M sodium hydroxide solution and the separated aqueous phase repeatedly extracted with DCM. The combined organic solutions were then dried over anhydrous magnesium sulfate, filtered, and evaporated in vacuo to afford 1-(benzylsulfonyl)-4-(dimethoxymethyl)benzene (3.3 g, 10.8 mmol, 27%).

¹H NMR (CDCl₃): 7.60-7.44 (4H, m, Ar), 7.24-7.17 (3H, m, Ar), 7.02-6.99 (2H, m, Ar), 5.38 (1H, s, CH(OMe)₂), 4.24 (2H, s, SO₂CH₂) and 3.24 (6H, s, CH(OMe)₂).

### Procedure D. (Acetal Deprotection): 4-(Benzylsulfonyl)benzaldehyde

A solution of 1-(benzylsulfonyl)-4-(dimethoxymethyl)benzene (3.33 g, 10.8 mmol) in THF (35 ml) was treated with an aqueous solution of sulfuric acid (2% v/v, 35 ml) and this was stirred at room temperature for about 18 hours. Excess potassium carbonate was then added to neutralise the solution, and the aqueous phase was extracted with DCM. The separated organic phase was dried over anyhydrous magnesium sulfate, filtered and evaporated to afford 4-(benzylsulfonyl)benzaldehyde as a white solid (1.62 g, 6.2 mmol, 57%)

¹H NMR (CDCl₃) 10.10 (1H, s, CHO), 7.98-7.92 (2H, d, J 8.4 Hz, Ar), 7.82-7.76 (2H, d, J 8.4 Hz, Ar), 7.38-7.23 (3H, m, Ar), 7.12-7.05 (2H, m, Ar) and 4.36 (2H, s, SO₂CH₂).

### Procedure E. (Reductive Alkylation): 2-(3-(4-(Benzylsulfonyl)benzyl)-5-fluoro-2-methyl-1H-indol-1-yl)acetic acid ethyl ester

4-(Benzylsulfonyl)benzaldehyde (1.62 g, 6.2 mmol) and 2-(5-fluoro-2-methyl-1H-indol-1-yl)acetic acid ethyl ester (1.34 g, 5.7 mmol) were dissolved in DCM (60 ml), and triethylsilane (4.53 ml, 28.5 mmol) added at room temperature. The solution was then cooled to 0° C. and trifluoroacetic acid (1.32 ml, 17.1 mmol) was added dropwise. Once addition was complete, the cooling bath was removed and the solution brought to room temperature where it was allowed to stir for 2 hours. The mixture was then partitioned by adding water, and the DCM layer was separated. The organic phase was evaporated and purified using flash column chromatography on silica gel (100% DCM was used as eluent) to afford 2-(3-(4-(benzylsulfonyl)benzyl)-5-fluoro-2-methyl-1H-indol-1-yl)acetic acid ethyl ester as a white solid (1.6 g, 3.3 mmol, 58% based on indole starting material).

¹H NMR (CDCl₃) 7.49 (2H, d, J 8.4 Hz, Ar), 7.36-7.18 (5H, m, Ar), 7.15-7.08 (1H, m, Ar), 7.05 (2H, d, J 8.5 Hz, Ar), 6.96-6.84 (2H, m, Ar), 4.80 (2H, s, NCH₂), 4.27 (2H, s, ArCH₂Ar) 4.22 (2H, q, J 7.1 Hz, CH₂CH₃), 4.10 (2H, s, SO₂CH₂), 2.33 (3H, s, ArCH₃) and 1.27 (3H, t, J 7.1 Hz, CH₂CH₃).

### Procedure F. (Saponification): 2-(3-(4-(Benzylsulfonyl)benzyl)-5-fluoro-2-methyl-1H-indol-1-yl)acetic acid (Compound 1)

To a solution of 2-(3-(4-(benzylsulfonyl)benzyl)-5-fluoro-2-methyl-1H-indol-1-yl)acetic acid ethyl ester (1.6 g, 3.3 mmol) in THF (10 ml) was added an aqueous solution of 1M potassium hydroxide (10 ml, 10 mmol) and the resulting mixture stirred vigorously for about 18 hours. THF was then removed in vacuo and the remaining solution acidified to pH 5.6 using 1N HCl. This was then extracted with DCM, and the separated organics dried over anhydrous magnesium sulfate, filtered and evaporated in vacuo. The resulting yellow solid was triturated with diethyl ether to afford 2-(3-(4-(benzylsulfonyl)benzyl)-5-fluoro-2-methyl-1H-indol-1-yl)acetic acid (1.05 g, 2.32 mmol, 70%).

¹H NMR (d₆ DMSO) 13.00 (1H, bs, CO₂H), 7.57 (2H, d, J 8.4 Hz, Ar), 7.40 (2H, d, J 8.4 Hz, Ar), 7.37-7.20 (4H, m, Ar), 7.13-7.08 (3H, m, Ar), 6.93-6.84 (1H, m, Ar), 4.97 (2H, s, NCH₂), 4.58 (2H, s, ArCH₂Ar) 4.12 (2H, S, SO₂CH₂) and 2.32 (3H, s, CH₃).

LCMS RT=4.02 mins (12.5 min run time), m/z MH⁺ 451.8.

2-(3-(4-(4-Chlorobenzylsulfonyl)benzyl)-5-fluoro-2-methyl-1H-indol-1-yl)acetic acid (Compound 2) was also prepared by this method except (4-chlorophenyl)methanethiol was used as the starting material in Procedure A.

¹H NMR (d₆ DMSO) 13.02 (1H, br s), 7.61-7.56 (2H, m), 7.45-7.33 (3H, m), 7.33-7.27 (2H, m), 7.13-7.06 (3H, m), 6.88 (1H, td, J 9.2 and 2.5), 4.97 (2H, s), 4.62 (2H, s), 4.12 (2H, s) and 2.32 (3H, s)

LCMS RT=2.48 mins (6.5 min run time), MH⁺ 485.7

2-(5-Fluoro-3-(4-(4-fluorobenzylsulfonyl)benzyl)-2-methyl-1H-indol-1-yl)acetic acid (Compound 6) was also prepared by this method except (4-fluorophenyl)methanethiol was used as the starting material in Procedure A.

¹H NMR (d₆ DMSO) 12.88 (1H, br s), 7.58 (2H, d, J 8.4), 7.41 (2H, d, J 8.4), 7.37 (1H, dd, J 8.8 and 4.3), 7.18-7.02 (5H, m), 6.88 (1H, td, J 9.2 and 2.5), 4.98 (2H, s), 4.61 (2H, s), 4.12 (2H, s), 2.32 (3H, s)

LCMS RT=2.40 mins (6.5 min run time), MH+ 469.8

### Synthesis of 2-(5-fluoro-3-(3-(4-fluorobenzylsulfonyl)benzyl)-2-methyl-1H-indol-1-yl)acetic acid (Compound 4)

The compound was synthesized according to the method set out in Scheme 3.

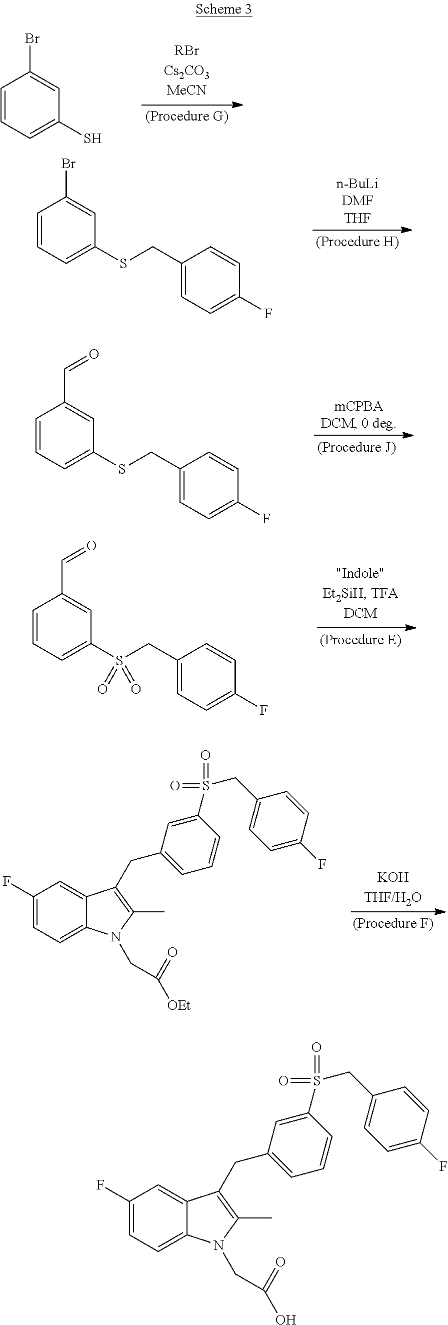

### Procedure G. (Alkylation): (3-Bromophenyl)(4-fluorobenzyl)sulfane

To a solution of 3-bromothiophenol (5 g, 26.4 mmol) and cesium carbonate (17.2 g, 52.8 mmol) in MeCN (75 ml) was added 4-fluorobenzyl bromide (3.57 ml, 29.1 mmol) over 10 minutes, and the resulting solution stirred at room temperature for 2 hours. The reaction was then quenched with water, and extracted with ethyl acetate. The separated organic phase was dried over anhydrous magnesium sulfate, filtered, and evaporated in vacuo to afford (3-bromophenyl)(4-fluorobenzyl)sulfane as an orange oil (8.85 g, quantitative yield).

¹H NMR (d₆ DMSO) 7.51 (1H, t, J 1.8 Hz, Ar), 7.43-7.31 (4H, m, Ar), 7.25 (1H, d, J 7.8 Hz, Ar), 7.18-7.11 (2H, m, Ar) and 4.29 (2H, s, SCH₂).

### Procedure H. (Formylation): 3-(4-Fluorobenzylthio)benzaldehyde

Anhydrous THF (50 ml) was purged with nitrogen, and cooled to −78° C. n-Butyllithium (16 ml, 2.5M in hexanes, 40 mmol) was added, and the solution allowed to stir for 15 minutes. To the resulting yellow/orange solution was added a solution of (3-bromophenyl)(4-fluorobenzyl)sulfane (7.86 g, 26.4 mmol) in anhydrous THF (20 ml) over 15 minutes at −78° C. Once addition was complete, this was stirred for a further 25 minutes before addition of anhydrous DMF (10 ml) in one portion. The resulting solution was stirred at −78° C. for 15 minutes, then allowed to warm to room temperature. After 45 minutes the reaction was quenched by the addition of water, and extracted with diethyl ether. The separated organic phase was washed with brine, then dried over anhydrous magnesium sulfate, filtered, and evaporated in vacuo. The resulting crude yellow oil (˜8 g) was purified using flash column chromatography on silica gel (gradient of 2-5% EtOAc in light petroleum as eluent) to afford 3-(4-fluorobenzylthio)benzaldehyde (4.15 g, 16.8 mmol, 64%).

¹H NMR (CDCl₃) 10.00 (1H, s, CHO), 7.79 (1H, t, J 1.5 Hz, Ar), 7.70-7.67 (1H, m, Ar), 7.53-7.50 (1H, m, Ar), 7.42 (1H, t, J 7.6 Hz, Ar), 7.35-7.28 (2H, m, Ar), 7.07-6.98 (2H, m, Ar) and 4.15 (2H, s, SCH₂).

### Procedure J. (Direct Oxidation): 3-(4-Fluorobenzylsulfonyl)benzaldehyde

To a solution of 3-(4-fluorobenzylthio)benzaldehyde (4.15 g, 16.8 mmol) in DCM (50 ml) at 0° C. was added mCPBA portionwise (˜75%, 11 g, 47.8 mmol). When addition was complete, the solution was allowed to warm to room temperature and stirred for about 18 hours. The resulting solution was then quenched with 1N NaOH and extracted with DCM. The combined organic phases were dried over anhydrous magnesium sulfate, filtered, and evaporated in vacuo. The resulting crude 3-(4-fluorobenzylsulfonyl)benzaldehyde (1.36 g, 4.9 mmol, 29%) was used directly without further purification.

¹H NMR (CDCl₃) 10.03 (1H, s, CHO), 8.19-8.12 (2H, m, Ar), 7.85 (1H, dt, J 8.0 and 1.5 Hz, Ar), 7.66 (1H, t, J 7.8 Hz, Ar), 7.12-7.06 (2H, m, Ar), 7.03-6.94 (2H, m, Ar) and 4.34 (2H, s, SCH₂).

### Procedure E. (Reductive Alkylation): 2-(5-Fluoro-3-(3-(4-fluorobenzylsulfonyl)benzyl)-2-methyl-1H-indol-1-yl)acetic acid ethyl ester

This reaction was carried out in a similar manner to Procedure E for Compound 1, except that the following reagent quantities were used:

3-(4-Fluorobenzylsulfonyl)benzaldehyde (1.36 g, 4.9 mmol);

2-(5-Fluoro-2-methyl-1H-indol-1-yl)acetic acid ethyl ester (1.12 g, 4.7 mmol);

DCM (20 ml);

Triethylsilane (4.06 ml, 25.5 mmol); and

TFA (1.1 ml, 14.3 mmol).

Following standard purification using flash column chromatography, 2-(5-fluoro-3-(3-(4-fluorobenzylsulfonyl)benzyl)-2-methyl-1H-indol-1-yl)acetic acid ethyl ester (500 mg, 1 mmol, 21% based on indole) was isolated.

¹H NMR (CDCl₃) 7.50-7.41 (3H, m, Ar), 7.38-7.30 (1H, m, Ar), 7.12 (1H, dd, J 8.6 and 4.2 Hz, Ar), 7.00-6.80 (6H, m, Ar), 4.81 (2H, s, NCH₂), 4.23 (2H, q, J 7.0 Hz, CH₂CH₃), 4.21 (2H, s, ArCH₂Ar), 4.05 (2H, S, SO₂CH₂), 2.32 (3H, s, ArCH₃) and 1.28 (3H, t, J 7.0 Hz, CH₂CH₃).

### Procedure F. (Saponification): 2-(5-Fluoro-3-(3-(4-fluorobenzylsulfonyl)benzyl)-2-methyl-1H-indol-1-yl)acetic acid

To a solution of 2-(5-fluoro-3-(3-(4-fluorobenzylsulfonyl)benzyl)-2-methyl-1H-indol-1-yl)acetic acid ethyl ester (500 mg, 1 mmol) in THF (5 ml) was added an aqueous solution of potassium hydroxide (169 mg in 5 ml water, 3 mmol), and this was stirred at room temperature for 2 hours. The reaction was then acidified with 2N HCl and extracted with DCM. The separated organic layer was dried over anhydrous magnesium sulfate, filtered, and evaporated in vacuo to give 2-(5-fluoro-3-(3-(4-fluorobenzylsulfonyl)benzyl)-2-methyl-1H-indol-1-yl)acetic acid as an orange solid (458 mg, 0.97 mmol, 97%).

¹H NMR (d₆ DMSO) 13.00 (1H, bs, CO₂H), 7.56 (1H, s, Ar), 7.53-7.43 (3H, m, Ar), 7.38 (1H, dd, J 9.0 and 4.5 Hz, Ar), 7.15-6.98 (5H, m, Ar), 6.88 (1H, td, J 9.1 and 2.5 Hz, Ar), 4.97 (2H, s, NCH₂), 4.63 (2H, s, ArCH₂Ar), 4.08 (2H, s, SO₂CH₂) and 2.29 (3H, s, CH₃)

LCMS RT=11.27 min (12.5 min run time), MH⁺ 469.8

2-(3-(3-(Benzylsulfonyl)benzyl)-5-fluoro-2-methyl-1H-indol-1-yl)acetic acid (Compound 3) was prepared using similar methods to those described for the synthesis of Compound 1 and Compound 4; specifically Procedures G, H, and B-F in that order, except benzyl bromide was used as the starting material in Procedure G.

¹H NMR (d₆ DMSO) 13.02 (1H, br s), 7.57-7.56 (1H, m), 7.51-7.42 (3H, m), 7.40-7.35 (1H, m), 7.30-7.16 (3H, m), 7.13-7.04 (3H, m), 6.88 (1H, td, J 9.2 and 2.4 Hz), 4.98 (2H, s), 4.61 (2H, s), 4.07 (2H, s) and 2.29 (3H, s)

LCMS RT=2.39 min (6.5 min run time), MH⁺ 452.4

### Synthesis of 2-(3-(2-(benzylsulfonyl)benzyl)-5-fluoro-2-methyl-1H-indol-1-yl)acetic acid (Compound 5)

The synthesis was carried out according to Scheme 4 below:

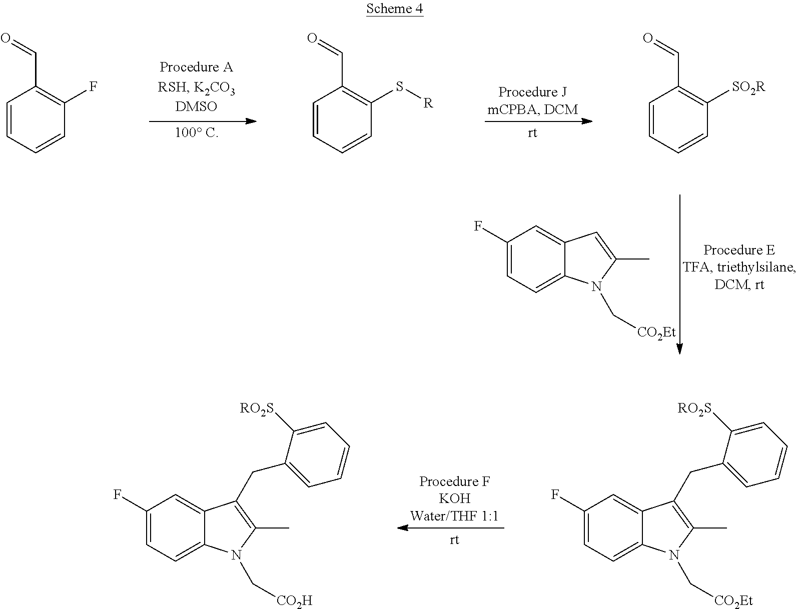

### Procedure A (SN_{A}r): 2-(Benzylthio)benzaldehyde

To a solution of phenylmethanethiol (1.5 g, 12.1 mmol) in DMSO (10 ml) was added potassium carbonate (4 g, 29 mmol) and 2-fluorobenzaldehyde (1.5 g, 12.1 mmol). This was heated to 100° C. for about 18 hours. The solution was then cooled to room temperature, poured onto water, and extracted with ethyl acetate. The aqueous layer was back extracted with further ethyl acetate, then the combined organic extracts were washed with water, dried over anhydrous magnesium sulfate, filtered and evaporated in vacuo to give a brown oil. This was purified using flash column chromatography on silica gel (eluting with a gradient of 0 to 30% ethyl acetate in light petroleum to afford 2-(benzylthio)benzaldehyde (0.38 g, 1.7 mmol, 14%).

Tlc (silica) Rf 0.76 (2:1 v/v light petroleum:ethyl acetate)

¹H NMR (d₆ DMSO) 10.12 (1H, s), 7.89-7.87 (1H, m), 7.62-7.59 (2H, m), 7.38-7.36 (3H, m), 7.34-7.31 (2H, m), 7.28-7.26 (1H, m), 4.27 (2H, s)

### Procedure J (Sulfide Oxidation): 2-(Benzylsulfonyl)benzaldehyde

Solid mCPBA (˜75%, 1.1 g, ˜4.8 mmol) was added slowly over 15 minutes to 2-(benzylthio)benzaldehyde (0.38 g, 1.7 mmol) in DCM (10 ml) at 0° C. Once addition was complete, the ice bath was removed, and the solution allowed to warm to room temperature and stirred for approximately 18 hours. The solution was then partitioned between 1M sodium hydroxide solution and ethyl acetate. The separated organic phase was then dried over anhydrous magnesium sulfate, filtered, and evaporated in vacuo to afford crude 2-(benzylsulfonyl)benzaldehyde (0.1 g, 0.38 mmol, 22%). This material was used directly in the next step without further purification.

Tlc (silica) Rf 0.5 (2:1 v/v light petroleum:ethyl acetate)

### Procedure E (Reductive Alkylation): 2-(3-(2-(Benzylsulfonyl)benzyl)-5-fluoro-2-methyl-1H-indol-1-yl)acetic acid ethyl ester

A mixture of 2-(benzylsulfonyl)benzaldehyde (0.1 g, 0.38 mmol) and 2-(5-fluoro-2-methyl-1H-indol-1-yl)acetic acid ethyl ester (91 mg, 0.38 mmol) were dissolved in DCM (5 ml), and triethylsilane (306 μL, 1.9 mmol) added at room temperature. Trifluoroacetic acid (90 μL, 1.17 mmol) was then added dropwise over 10-15 minutes. Once addition was complete the solution was stirred at room temperature for about 18 hours. The mixture was then neutralised by adding aqueous NaHCO₃ solution, and the organic layer diluted with additional DCM. The organic phase was then separated, dried over anhydrous magnesium sulfate, filtered and evaporated in vacuo. The residue was purified using flash column chromatography on silica gel (a gradient of 0 to 40% ethyl acetate in light petroleum was used as eluent) to afford 2-(3-(2-(benzylsulfonyl)benzyl)-5-fluoro-2-methyl-1H-indol-1-yl)acetic acid ethyl ester (80 mg, 167 μmol, 44% based on indole starting material).

¹H NMR (CDCl₃): 7.74 (1H, dd, J 7.8 1.2 Hz), 7.43-7.21 (5H, m), 7.16-7.08 (2H, m), 7.03-6.99 (2H, m), 6.93-6.82 (2H, m), 4.80 (2H, s), 4.36 (2H, s), 4.27 (2H, s), 4.21 (2H, q, J 7.5 Hz), 2.31 (3H, s), 1.27 (3H, t, J 7.5 Hz)

LCMS RT=6.84 min (12.5 min run time), MH⁺ 479.8;

### Procedure F (Saponification): 2-(3-(2-(Benzylsulfonyl)benzyl)-5-fluoro-2-methyl-1H-indol-1-yl)acetic acid

To a solution of 2-(3-(2-(benzylsulfonyl)benzyl)-5-fluoro-2-methyl-1H-indol-1-yl)acetic acid ethyl ester (80 mg, 167 μmol) in THF (5 ml) was added an aqueous solution of potassium hydroxide (47 mg in 5 ml of water, 0.84 mmol) and the resulting mixture stirred for 3 hours. The THF was then removed in vacuo and the remaining solution acidified using 2N HCl. This was then extracted with ethyl acetate, and the separated organics dried over anhydrous magnesium sulfate, filtered and evaporated in vacuo. The resulting solid was dried in a vacuum oven to afford 2-(3-(2-(benzylsulfonyl)benzyl)-5-fluoro-2-methyl-1H-indol-1-yl)acetic acid in quantitative yield.

¹H NMR (d₆ DMSO): 13.00 (1H, br), 7.68 (1H, dd, J 8.0 1.3 Hz), 7.48 (1H, td, J 7.5 1.6 Hz), 7.39 (1H, dd, J 9.0 4.4 Hz), 7.36-7.26 (4H, m), 7.17-7.14 (2H, m), 7.05-7.00 (1H, m), 6.96-6.84 (2H, m), 5.00 (2H, s), 4.68 (2H, s), 4.42 (2H, s), 2.26 (3H, s)

LCMS RT=4.78 min (12.5 min run time), M−H⁺ 450.1

### EXAMPLE 2

### Measurement of CRTH2 Antagonist Activity

Materials and Methods

Materials

Mono-poly resolving medium was obtained from Dainippon Pharmaceuticals (Osaka, Japan). Macs anti-CD16 microbeads were from Miltenyi biotec (Bisley, Surrey). ChemoTx plates were purchased from Neuroprobe (Gaithersburg, Md.). Poly-D-lysine coated 96-well plates were obtained from Greiner (Gloucestershire, UK). [³H]PGD₂ was from Amersham Biosciences (Buckinghamshire, UK). [³H]SQ29548 was purchased from Perkin Elmer Life Sciences (Buckinghamshire, UK). All other reagents were obtained from Sigma-Aldrich (Dorset, UK), unless otherwise stated.

Methods

Cell Culture

Chinese Hamster Ovary cells were transfected with CRTH2 or DP receptors (CHO/CRTH2 and CHO/DP) and were maintained in culture in a humidified atmosphere at 37° C. (5% CO₂) in Minimum Essential Medium (MEM) supplemented with 10% foetal bovine serum, 2 mM glutamine, and 1 mg ml⁻¹ active G418. The cells were passaged every 2-3 days. For radioligand binding assay, cells were prepared in triple-layer flasks or in 175 cm² square flasks (for membrane preparation).

Preparation of Cell Membranes

Membranes were prepared either from CHO/CRTH2 and CHO/DP cells, or from platelets (as a source of TP receptors). CHO cells grown to confluency were washed with PBS and detached using a Versene solution (15 ml per flask). When the cells were grown in 175 cm² square flask, they were collected by scrapping in PBS. The cell suspensions were centrifuged (1,700 rpm, 10 min, 4° C.) and resuspended in 15 ml of buffer (1× HBSS, supplemented with 10 mM HEPES, pH 7.3). Cell suspensions were then homogenised using an Ultra Turrax at setting 4-6 for 20 s. The homogenate was centrifuged at 1,700 rpm for 10 min and the supernatant was collected and centrifuged at 20,000 rpm for 1 h at 4° C. The resulting pellet was resuspended in buffer and stored at −80° C. in aliquots of 200-500 μl. The protein concentration was determined by the method of Bradford (1976), using bovine serum albumin as standard. The platelets were washed by centrifugation at 600×g for 10 min and resuspended in ice-cold assay buffer (10 mM Tris-HCl, pH 7.4, 5 mM Glucose, 120 mM NaCl, 10 μM indomethacin) and directly centrifuged at 20,000 rpm for 30 min at 4° C. The resulting pellet was treated as described above.

Radioligand Binding Assays

[³H]PGD₂ (160 Ci/mmol) binding experiments were performed on membranes prepared as described above. Assays were performed in a final volume of 100 μl of buffer (1× HBSS/HEPES 10 mM, pH 7.3). Cell membranes (15 μg) were preincubated at room temperature with varying concentration of competing ligand for 15 min. [³H]PGD₂ was then added and the incubation continued for a further one hour at room temperature. The reaction was terminated by the addition of 200 μl ice-cold assay buffer to each well, followed by rapid filtration through Whatman GF/B glass fibre filters using a Unifilter Cell harvester (PerkinElmer Life Sciences) and six washes of 300 μl of ice-cold buffer. The Unifilter plates were dried at room temperature for at least 1 h and the radioactivity retained on the filters was determined on a Beta Trilux counter (PerkinElmer Life Sciences), following addition of 40 μl of Optiphase Hi-Safe 3 (Wallac) liquid scintillation. Non specific binding was defined in the presence of 10 μM unlabelled PGD₂. Assays were performed in duplicate.

The results of the radioligand binding experiments to the CRTH2 are shown in Table 1.

TABLE 1
Radioligand binding data (Kᵢ on CRTH2 Receptor).

| Compound No. | Kᵢ (nM) |
|---|---|
| 1 | 1 |
| 2 | 5 |
| 3 | 3 |
| 4 | 1 |
| 5 | 3 |
| 6 | 3 |

### EXAMPLE 3

### Human Whole Blood Eosinophil Shape Change Assay

Compounds 1-6 were assayed for their effect on PGD₂ induced eosinophil shape change.

Methods

Shape Change Assay in Whole Blood

Compounds (1 μl, 200× final concentration) were added directly to 200 μl whole blood, mixed well and incubated for 15 min, 37° C., 5% CO₂. After this time, cell shape was fixed by addition of 300 μl Cytofix™ buffer (BD Biosciences), 15 min on ice. 10 ml RBC lysis buffer was added to the fixed cells, incubated 5 min, at room temperature and centrifuged, 300×g for 5 min. Supernatant (containing lysed red cells) was removed and the lysis step was repeated. Leukocytes were resuspended in 250 μl RPMI/10% FCS and shape change analysed by FACS. Eosinophils were gated out based on their autofluorescence and 2000 eosinophil events were counted per sample. Data were analysed in triplicate. The results for the eosinophil shape change assay are shown in Table 2.

TABLE 2
IC₅₀ Values for the Effect of Test Compounds on 10 nM
PGD₂-induced Eosinophil Shape Change in whole blood

| Compound No. | Value (nM) |
|---|---|
| 1 | 1 |
| 3 | 77 |
| 5 | 12 |
| 6 | 3 |

Compounds which are suitable for use as pharmaceutical agents generally have an IC₅₀ value in the eosinophil shape change test of between about 1 and 10 nM. However, Compounds 1 and 6 are some of the most active compounds we have tested, and have similar or greater activity than the compounds of WO2008/012511 as Example Compounds 1 to 3 of that document have IC₅₀ values in the above test of 5 nM, 2 nM and 6 nM respectively.

It is also surprising, in view of the teaching of that document, that the most active compounds of the present invention are those in which the group Z is at the 4-position. In WO2008/012511, Comparator Compounds C, D and E, which are the 4-regioisomers of Compounds 1 to 3 had IC₅₀ values in the above test of 273 nM, 494 nM and 71 nM respectively and are therefore considerably less active in whole blood than either the compounds of WO2008/012511 or the more active compounds of the present invention.

1. A compound of general formula (I) 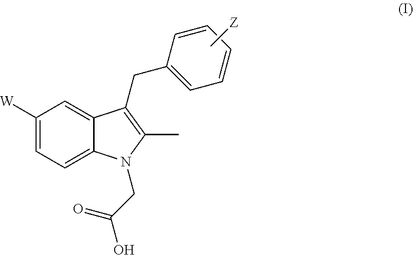
wherein
W is chloro or fluoro;
Z is a —SO₂YR¹ group wherein R¹ is C₃-C₈ heterocyclyl, aryl or heteroaryl any of which may optionally be substituted with one or more substituents selected from halo, —CN, —C₁-C₆ alkyl, —SOR³, —SO₂R³, —SO₂N(²)₂, —N(R²)², —NR²C(O)R³, —CO₂R², —CONR²R³, —NO₂, —OR², —SR₂, —O(CH₂)ₚOR², or —O(CH₂)ₚO(CH₂)_{q}OR² wherein
each R² is independently hydrogen, —C₁-C₆ alkyl, —C₃-C₈ cycloalkyl, aryl or heteroaryl;
each R³ is independently, —C₁-C₆ alkyl, —C₃-C₈ cycloalkyl, aryl or heteroaryl;
p and q are each independently an integer from 1 to 3; and
Y is a straight or branched C₁-C₄ alkylene chain;
or a pharmaceutically acceptable salt, hydrate, solvate, complex or prodrug thereof.

2. A compound of general formula (II): 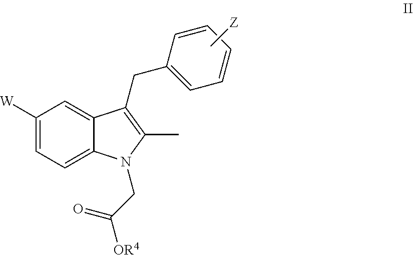
wherein
W is chloro or fluoro;
Z is a —SO₂YR¹ group wherein R¹ is C₃-C₈ heterocyclyl, aryl or heteroaryl any of which may optionally be substituted with one or more substituents selected from halo, —CN, —C₁-C₆ alkyl, —SOR³, —SO₂R³, —SO₂N(R²)₂, —N(R²)₂, —NR²C(O)R³, —CO₂R², —CONR²R³, —NO₂, —OR², —SR², —O(CH₂)ₚOR², or —O(CH₂)ₚO(CH₂)_{q}OR² wherein
each R² is independently hydrogen, —C₁-C₆ alkyl, —C₃-C₈ cycloalkyl, aryl or heteroaryl;
each R³ is independently, —C₁-C₆ alkyl, —C₃-C₈ cycloalkyl, aryl or heteroaryl;
p and q are each independently an integer from 1 to 3;
Y is a straight or branched C₁-C₄ alkylene chain;
R⁴ is C₁-C₆ alkyl, C₁-C₆ alkyl substituted with aryl, aryl, (CH₂)ₘOC(═O)C₁-C₆alkyl, ((CH₂)ₘO)ₙCH₂CH₂X, (CH₂)ₘN(R⁵)₂ or CH((CH₂)ₘO(C═O)R⁶)₂;
m is 1 or 2;
n is 1-4;
X is OR⁵ or N(R⁵)₂;
R⁵ is hydrogen or methyl; and
R⁶ is C₁-C₁₈ alkyl;
or a pharmaceutically acceptable salt, hydrate, solvate, complex or prodrug thereof.

3. A compound as claimed in claim 1 or claim 2, wherein W is fluoro.

4. A compound as claimed in claim 1 or claim 2, wherein R¹ is a phenyl group which is unsubstituted or is substituted with a single halo substituent.

5. A compound as claimed in claim 4, wherein the halo substituent is fluoro or chloro.

6. A compound as claimed in claim 5, wherein the halo substituent is at the 4-position of the phenyl group R¹.

7. A compound as claimed in claim 1 or claim 2, wherein Y is methylene.

8. A compound as claimed in claim 1 or claim 2, wherein Z is at the 4-position of the benzyl group.

9. A compound as claimed in claim 1 selected from the group consisting of:
2-(3-(4-(Benzylsulfonyl)benzyl)-5-fluoro-2-methyl-1H-indol-1-yl)acetic acid;
2-(3-(4-(4-Chlorobenzylsulfonyl)benzyl)-5-fluoro-2-methyl-1H-indol-1-yl)acetic acid;
2-(3-(3-(Benzylsulfonyl)benzyl)-5-fluoro-2-methyl-1H-indol-1-yl)acetic acid;
2-(5-Fluoro-3-(3-(4-fluorobenzylsulfonyl)benzyl)-2-methyl-1H-indol-1-yl)acetic acid;
2-(3-(2-(Benzylsulfonyl)benzyl)-5-fluoro-2-methyl-1H-indol-1-yl)acetic acid;
2-(3-(4-(4-Fluorobenzylsulfonyl)benzyl)-5-fluoro-2-methyl-1H-indol-1-yl)acetic acid;
and the C₁-C₆ alkyl, aryl, (CH₂)ₘOC(═O)C₁-C₆alkyl, ((CH₂)ₘO)ₙCH₂CH₂X, (CH₂)ₘN(R⁵)₂ or CH((CH₂)ₘO(C═O)R⁶)₂ esters thereof; wherein
m is 1 or 2;
n is 1-4;
X is OR⁵ or N(R⁵)₂;
R⁵ is hydrogen or methyl; and
R⁶ is C₁-C₁₈ alkyl.

10. A process for the preparation of a compound of general formula (I) as claimed in claim 1, the process comprising reacting with a base, a compound of general formula (II), 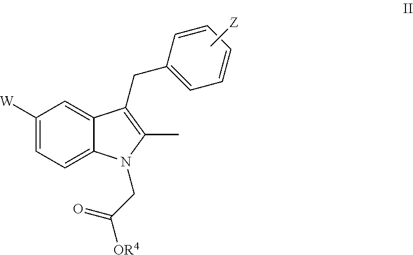
wherein
W and Z are as defined in claim 1, and
R⁴ is C₁-C₆ alkyl.

11. A method for the treatment of a disease or condition mediated by PGD₂ or other agonists at the CRTH2 receptor, the method comprising administering to a patient in need of such treatment a suitable amount of a compound as claimed in any one of claims 1 and 2.

12. A method as claimed in claim 11, wherein the disease or condition is asthma, including allergic asthma, bronchial asthma, exacerbations of asthma and related allergic diseases caused by viral infection, particularly those exacerbations caused by rhinovirus and respiratory syncytial virus intrinsic, extrinsic, exercise-induced, drug-induced and dust-induced asthma, treatment of cough, including chronic cough associated with inflammatory and secretory conditions of the airways and iatrogenic cough, acute and chronic rhinitis, including rhinitis medicamentosa, vasomotor rhinitis, perennial allergic rhinitis, seasonal allergic rhinitis, nasal polyposis, acute viral infection including common cold, infection due to respiratory syncytial virus, influenza, coronavirus and adenovirus, atopic dermatitis, contact hypersensitivity (including contact dermatitis), eczematous dermatitis, phyto dermatitis, photo dermatitis, sebhorroeic dermatitis, dermatitis herpetiformis, lichen planus, lichen sclerosis et atrophica, pyoderma gangrenosum, skin sarcoid, discoid lupus erythematosus, pemphigus, pemphigoid, epidermolysis bullosa urticaria, angioedema, vasculitides, toxic erythemas, cutaneous eosinophilias, alopecia areata, male-pattern baldness, Sweet's syndrome, Weber-Christian syndrome, erythema multiforme, cellulitis, panniculitis, cutaneous lymphomas, non-melanoma skin cancer and other dysplastic lesions; blepharitis conjunctivitis, especially allergic conjunctivitis, anterior and posterior uveitis, choroiditis, autoimmune, degenerative or inflammatory disorders affecting the retina, ophthalmitis; bronchitis, including infectious and eosinophilic bronchitis, emphysema, bronchiectasis, farmer's lung, hypersensitivity pneumonitis, idiopathic interstitial pneumonias, complications of lung transplantation, vasculitic and thrombotic disorders of the lung vasculature, pulmonary hypertension, food allergies, gingivitis, glossitis, periodontitis, oesophagitis including reflux, eosinophilic gastroenteritis, proctitis, pruris ani, celiac disease, food-related allergies, inflammatory bowel disease, ulcerative colitis and Crohn's disease, mastocytosis and also other CRTH2-mediated diseases, for example autoimmune diseases such as hyper IgE syndrome, Hashimoto's thyroiditis, Graves' disease, Addison's disease, diabetes mellitus, idiopathic thrombocytopaenic purpura, eosinophilic paschiitis, antiphospholipid syndrome and systemic lupus erythematosus, AIDS, leprosy, Sezary syndrome, paraneoplastic syndrome, mixed and undifferentiated connective tissue diseases, inflammatory myopathies including dermatomyositis and polymyositis, polymalgia rheumatica, juvenile arthritis, rheumatic fever, vasculitides including giant cell arteritis, Takayasu's arteritis, Churg-Strauss syndrome, polyarteritis nodosa, microscopic polyarteritis, temporal arteritis, myasthenia gravis, acute and chronic pain, neuropathic pain syndromes, central and peripheral nervous system complications of malignant, infectious or autoimmune processes, low back pain, familial Mediterranean Fever, Muckle-Wells syndrome, Familial Hibernian fever, Kikuchi disease, psoriasis, acne, multiple sclerosis, allograft rejection, reperfusion injury, chronic obstructive pulmonary disease, as well as rheumatoid arthritis, Still's disease, ankylosing spondylitis, reactive arthritis, undifferentiated spondarthropathy, psoriatic arthritis, septic arthritis and other infection-related arthopathies and bone disorders and osteoarthritis; acute and chronic crystal-induced synovitis including urate gout, calcium pyrophosphate deposition disease, calcium paptite related tendon syndrome and synovial inflammation, Behcet's disease, primary and secondary Sjogren's syndrome systemic sclerosis and limited scleroderma; hepatitis, cirrhosis of the liver, cholecystitis, pancreatitis, nephritis, nephritic syndrome, cystitis and Hunner's ulcer, acute and chronic urethritis, prostatitis, epididymitis, oophoritis, salpingitis, vulvo-vaginitis, Peyronie's disease, erectile dysfunction, Alzheimer's disease and other dementing disorders; pericarditis, myocarditis, inflammatory and auto-immune cardiomyopathies including myocardial sarcoid, ischaemic reperfusion injuries, endocarditis, valvulitis, aortitis, phlebitis, thrombosis, treatment of common cancers and fibrotic conditions such as idiopathic pulmonary fibrosis including cryptogenic fibrosing alveolitis, keloids, excessive fibrotic scarring/adhesions post surgery, liver fibrosis including that associated with hepatitis B and C, uterine fibroids, sarcoidosis, including neurosarcoidosis, scleroderma, kidney fibrosis resulting from diabetes, fibrosis associated with RA, atherosclerosis, including cerebral atherosclerosis, vasculitis, myocardial fibrosis resulting from myocardial infarction, cystic fibrosis, restenosis, systemic sclerosis, Dupuytren's disease, fibrosis complicating anti-neoplastic therapy and chronic infection including tuberculosis and aspergillosis and other fungal infections, CNS fibrosis following stroke or the promotion of healing without fibrotic scarring.

13. A method as claimed in claim 11, wherein the condition to be treated or prevented is allergic asthma, perennial allergic rhinitis, seasonal allergic rhinitis, atopic dermatitis, contact hypersensitivity, conjunctivitis, eosinophilic bronchitis, food allergies, eosinophilic gastroenteritis, inflammatory bowel disease, ulcerative colitis, Crohn's disease, mastocytosis, autoimmune disease, psoriasis, acne, multiple sclerosis, allograft rejection, reperfusion injury, chronic obstructive pulmonary disease, rheumatoid arthritis, psoriatic arthritis, osteoarthritis, fibrotic diseases caused/exacerbated by Th2 immune responses, idiopathic pulmonary fibrosis or hypertrophic scars.

14. A method as claimed in claim 11 further comprising administering one or more additional active agents selected from the group consisting of:
other CRTH2 antagonists, Suplatast tosylate, β2 adrenoreceptor agonists, methylxanthines, mast cell stabilisers, muscarinic receptor antagonists, antihistamines, α₁ and α₂ adrenoreceptor agonists, modulators of chemokine receptor function, leukotriene antagonists, leukotriene biosynthesis inhibitors, 5-lipoxygenase activating protein inhibitors, N-(5-substituted)-thiophene-2-alkylsolfonamides, 2,6-di-tert-butylphenol hydrazones, methoxytetrahydropyrans, pyridinyl-substituted-2-cyanonaphthalene compounds, 2-cyanoquinoline compounds, indole and quinoline compounds, phosphodiesterase inhibitors, anti-IgE antibody therapies, anti-infectives, anti-fungals, immunosuppressants, immunotherapy agents, corticosteroids, drugs which promote Th1 cytokine response, other antagonists of PGD₂ acting at other receptors such as DP antagonists, drugs that modulate cytokine production, TNF receptor immunoglobulin molecules, inhibitors of other TNF isoforms, non-selective COX-1/COX-2 inhibitors, COX-2 inhibitors, low dose methotrexate, lefunomide, ciclesonide, hydroxychloroquine, d-penicillamine, auranofin, parenteral or oral gold, drugs that modulate the activity of Th2 cytokines IL-4 and IL-5, PPAR-γ agonists, anti-RSV antibodies and agents that may be used to treat rhinovirus infection.

15. A pharmaceutical composition comprising a compound as claimed in claim 1 or claim 2 together with a pharmaceutical excipient or carrier.

16. A composition as claimed in claim 15 formulated for oral, rectal, nasal, bronchial, topical, vaginal or parenteral administration.

17. A composition as claimed in claim 15 further comprising one or more additional active agents useful in the treatment of diseases and conditions mediated by PGD₂ or other agonists at the CRTH2 receptor.

18. A composition as claimed in claim 17, wherein the additional active agents are selected from a group consisting of:
other CRTH2 antagonists, Suplatast tosylate, β2 adrenoreceptor agonists, methylxanthines, mast cell stabilisers, muscarinic receptor antagonists, antihistamines, α₁ and α₂ adrenoreceptor agonists, modulators of chemokine receptor function, leukotriene antagonists, leukotriene biosynthesis inhibitors, 5-lipoxygenase activating protein inhibitors, N-(5-substituted)-thiophene-2-alkylsolfonamides, 2,6-di-tert-butylphenol hydrazones, methoxytetrahydropyrans, pyridinyl-substituted-2-cyanonaphthalene compounds, 2-cyanoquinoline compounds, indole and quinoline compounds, phosphodiesterase inhibitors, anti-IgE antibody therapies, anti-infectives, anti-fungals, immunosuppressants, immunotherapy agents, corticosteroids, drugs which promote Th1 cytokine response, other antagonists of PGD₂ acting at other receptors such as DP antagonists, drugs that modulate cytokine production, TNF receptor immunoglobulin molecules, inhibitors of other TNF isoforms, non-selective COX-1/COX-2 inhibitors, COX-2 inhibitors, low dose methotrexate, lefunomide, ciclesonide, hydroxychloroquine, d-penicillamine, auranofin, parenteral or oral gold, drugs that modulate the activity of Th2 cytokines IL-4 and IL-5, PPAR-γ agonists, anti-RSV antibodies and agents that may be used to treat rhinovirus infection.

19. A process for the preparation of a pharmaceutical composition comprising bringing a compound of claim 1 or claim 2 in conjunction or association with a pharmaceutically or veterinarily acceptable carrier or vehicle.

20. A kit for the treatment of a disease or condition mediated by the action of PGD₂ at the CRTH2 receptor comprising,
(a) a first container comprising a compound of claim 1 or claim 2 and
(b) a second container comprising an additional agent useful for the treatment of diseases and conditions mediated by PGD₂ or other agonists at the CRTH2 receptor.

21. The kit as claimed in claim 20 wherein said additional agent is selected from the group consisting of:
other CRTH2 antagonists, Suplatast tosylate, β2 adrenoreceptor agonists, methylxanthines, mast cell stabilisers, muscarinic receptor antagonists, antihistamines, α₁ and α₂ adrenoreceptor agonists, modulators of chemokine receptor function, leukotriene antagonists, leukotriene biosynthesis inhibitors, 5-lipoxygenase activating protein inhibitors, N-(5-substituted)-thiophene-2-alkylsolfonamides, 2,6-di-tert-butylphenol hydrazones, methoxytetrahydropyrans, pyridinyl-substituted-2-cyanonaphthalene compounds, 2-cyanoquinoline compounds, indole and quinoline compounds, phosphodiesterase inhibitors, anti-IgE antibody therapies, anti-infectives, anti-fungals, immunosuppressants, immunotherapy agents, corticosteroids, drugs which promote Th1 cytokine response, other antagonists of PGD₂ acting at other receptors such as DP antagonists, drugs that modulate cytokine production, TNF receptor immunoglobulin molecules, inhibitors of other TNF isoforms, non-selective COX-1/COX-2 inhibitors, COX-2 inhibitors, low dose methotrexate, lefunomide, ciclesonide, hydroxychloroquine, d-penicillamine, auranofin, parenteral or oral gold, drugs that modulate the activity of Th2 cytokines IL-4 and IL-5, PPAR-γ agonists, anti-RSV antibodies and agents that may be used to treat rhinovirus infection.
